(19) United States
(12) Patent Application Publication
(10) Pub. No.: US 2020/0352765 A1
(43) Pub. Date: Nov. 12, 2020

(54) Oral Appliance As Swallow Training Auxiliary Device With Open Occlusal Surface Of Teeth And Production Method Thereof

(71) Applicant: Yi-Yueh Lin, KaoSiung City (TW)

(72) Inventor: Yi-Yueh Lin, KaoSiung City (TW)

(21) Appl. No.: 16/869,605

(22) Filed: May 8, 2020

(30) Foreign Application Priority Data
May 8, 2019 (TW) 108115917

(51) Int. Cl. A61F 5/00; A61C 5/00

(52) U.S. Cl. CPC A61F 5/00; A61C 5/007

(57) ABSTRACT

An oral appliance as swallow training auxiliary device with open occlusal surface includes an open fixing frame and a palate pad. The open fixing frame is first made from cast by elastic material, or other elastic material done by 3D printed technology, and the palate pad uses an unsetting soft material to make a palate pad. After placement of the open fixing frame with the palate pad template on the upper dental arch of a patient, the patient does swallowing and uses tongue to press up on and cause the palate pad template to mold the shape of the palate pad to match tongue elevation position during swallowing and palate side in the oral cavity of the patient.

### CROSS-REFERENCES TO RELATED APPLICATIONS

This application claims priority from Taiwanese Application Serial Number 108115917 filed May 8, 2019, which is hereby incorporated herein by reference in its entirety.

### BACKGROUND OF THE INVENTION

### (A) Field of the Invention

The present invention relates to an oral appliance as swallow training auxiliary device with open occlusal surface and production method thereof, and more particularly to a training auxiliary device that is characterized in being mounted on the teeth of upper dental arch to enable exposure of the occlusal surface of teeth thereof.

### (b) Description of the Prior Art

The phases involved in normal swallowing process include the oral phase, pharyngeal phase, and the esophageal phase, whereby saliva or chewed food is caused to enter the stomach through the coordinated application of action and force of muscles in each phase of the swallowing process. When swallowing in the oral phase, the tongue presses up on the upper palate that produces a pressure, people with well preformed swallowing function can achieving a maximum tongue pressure of 30 kPa to 90 kPa or higher. The hope is that old people can also achieve a preferred maximum tongue pressure of more than 30 kPa to enable performing the optimum swallowing function cause saliva, liquid, and bolus formed from saliva and chewed food can be well pressed to the pharynx in order to activate the subsequent swallowing reflex processes.

In a situation whereby a patient is unable to swallow normally, which is medically known as dysphagia, and symptoms of dysphagia occurs in the oral phase include: inadequate tongue movement occurs during chewing and swallowing, two to three swallows of each mouthful of food are required in order to complete swallowing of all the food, sand food has accumulated as debris residue on palate, on the tongue surface, and on the mucosa of the oral cavity after having eaten a meal, usually cause poor oral hygiene. The main factor resulting in the above-described symptoms derives from the patient effecting inadequate pressure when their tongue presses up on the upper palate and prepares to use a pressure difference to cause food bolus in oral cavity to enter the pharynx. Actual measurement of maximum tongue pressure of patients with dysphagia is generally an inadequate pressure, may below 30 kPa, even below 20 kPa. Dysphagia easily occurs if the muscles of oral function used in swallowing are weakened, especially muscle of tongue.

Thus, in order to help patients restore their swallowing function, treatment methods include direct swallowing rehabilitation training, it means patient swallow saliva, modified liquid, or modified food during training, and we can use of an auxiliary device for better swallowing progress. Such as tongue holding method, the patient can do a training to exercise as tightly closes their two lips to hold the protruded tongue and do saliva swallowing, that can enhance the endurance and power of tongue muscle. The oral appliance can be an auxiliary device to help the saliva swallowing progress. Whereby the swallowing ability of the patient can be restored progressively by the swallow training.

An auxiliary device is mainly a palate pad placed on the upper palate in the oral cavity of a patient, which shortens the distance between the tongue and the upper palate, makes the cavity space smaller, the thereby enabling the tongue to apply a greater pressure (greater is better for well performed swallowing function) on the upper palate and enable swallowing to smoothly progress in the oral cavity section. A patient trains with the auxiliary device placed in their oral cavity, the power and endurance of tongue muscle may increase progressively. After a period of time of training, around two to three months, we can measures whether or not the patient's maximum tongue pressure is larger than 30 kPa without wearing the oral appliance as auxiliary device, and evaluates whether or not the patient no longer needs to wear the auxiliary device and is able to swallow by themselves. One type of oral appliance device, as shown in FIGS. 10 and 11, is formed as a palate pad (A) with a thickness (H), which is hooked in place on the teeth by means of fixing hooks (B), thereby fixing the palate pad (A) in the oral cavity. However, the oral cavity shape of the patient (such as the gum shape asymmetry, tooth axis may be tilting, dental ill alignment) is not generally completely symmetrical, having portions that are crooked. And conventional fixed hooks and palate pad material has poor elasticity, thus unable to adequately deform in the placement procedure to accommodate the oral cavity shape of different patients. Sometimes, some teeth need to be extracted in order to accommodate the path of insertion for placement procedure of the auxiliary device. In addition, when the occlusal surface of the teeth of the upper dental arch and the occlusal surface of the teeth of lower dental arch proceed with occlusal action, the fixing hooks (B) used to hook onto the occlusal surface of the teeth create an hindrance, after multiple reuse a period of time, the fixing hooks (B) may possibly break and come apart, and may cause injuries to the patient, even inadvertently swallowing of the fixing hooks. Another type of oral appliance as auxiliary device, as shown in FIGS. 12 and 13, is similar to the shape of a complete denture or partial denture and includes a mounting portion (C) and a palate pad (D). The palate pad (D) has an augmentation thickness (H′) and is mounted onto the teeth using the mounting portion (C), thereby fixing the auxiliary device in the oral cavity. However, this type of auxiliary device has the same shortcoming of being made from material of poor elasticity, and thus unable to adequately deform to accommodate the oral cavity shape of different patients, resulting in the problem of having a poor fastening effect. Moreover, because this type of auxiliary device is mounted on the denture, the occlusal force on the teeth of denture make the device move unstable, especially when the patient's occlusion is malocclusion, which affects the patient's willingness to use and degree of comfortability when in use.

### SUMMARY OF THE INVENTION

Based on the above-described shortcomings of auxiliary devices of the prior art, the present invention provides an oral appliance as swallow training auxiliary device with open occlusal surface thereof, which includes an open fixing frame and a palate pad, wherein:

The open fixing frame generally an teeth arch from brace, which includes a mutually connected outer edge and an inner edge, with an open space defined therebetween that enables exposing the occlusal surface of upper dental arch. The palatal augmentation pad corresponds in shape of palatal side of the oral cavity; moreover, the palate pad is fixed to the inner edge.

Preferably, the outer edge includes an outer edge binding surface, which corresponds to an arranged configuration of outer surface of the teeth of upper dental arch, and the inner edge includes an inner edge binding surface, which corresponds to an arranged configuration of an inner surface of the teeth of upper dental arch.

Preferably, the open fixing frame and baseplate of palate pad are made from elastic material, such as thin thermoplastic resin plate, and the palate pad is modified and finish the form by soft material, such as denture soft liner.

Preferably, an auxiliary binding structure is disposed on the inner edge, and the palate pad is fixed to the inner edge by means of the auxiliary binding structure, with the auxiliary binding structure provided with several through holes.

Preferably, the palate pad includes: a baseplate and colloid layers respectively coated on two opposite surfaces of the baseplate. The open fixing frame and the baseplate are made from elastic material, such as thin thermoplastic resin plate, and the colloid layers are made from soft material, such as denture soft liner.

Preferably, the open fixing frame is only mounted on the teeth of upper dental arch, or the open fixing frame is further mounted on the gum of the upper jaw.

The present invention further provides a production method for the oral appliance as swallow training auxiliary device with open occlusal surface, including the following steps:

Producing the open fixing frame according to the arranged configuration of the teeth of upper dental arch, wherein the open fixing frame includes the mutually connected outer edge and an inner edge, with the open space being formed between the outer edge and the inner edge that enables exposing the occlusal surface of the teeth of upper dental arch. Producing the palate pad on the inner edge to match the palate side in the oral cavity.

Preferably, producing the open fixing frame according to the arranged configuration of the upper dental arch and the shape of the gum on the upper jaw.

Preferably, the open fixing frame is produced by applying elastic material, such as thin thermoplastic resin plate on the teeth of upper dental arch of a dental mold.

Alternatively, the open fixing frame is produced by firstly obtaining 3D digital data of the upper dental arch and gum of upper jaw, and then depicting and shaping the open fixing frame with a 3D printing to technology.

Preferably, a palatal augmentation palate pad is produced using an unsetting soft material, such as denture soft liner, on the palatal baseplate template, connected to the inner edge. After fitting the open fixing frame in the patient's oral cavity, the patient do swallow and uses their tongue to push up on and to shaped the form of the palatal augmentation palate pad. The palate pad baseplate template is produced by first forming a baseplate on the inner edge of the open fixing frame, and then the two opposite surfaces of the baseplate are coated both with the unsetting soft material, such as denture soft liner.

Based on the above-described technological characteristics, the present invention is preferably able to achieve the following effects:
1. Mounting of the open fixing frame on the teeth of upper dental arch enables exposing the occlusal surface thereof. Accordingly, a patient still directly uses the occlusal surface of the upper teeth to produce occlusion whether speaking or chewing food, which is equivalent to the occlusal habit of the average person, thereby increasing the degree of comfort when in use the device, improving the patient's willingness to use the device.
2. The present invention is mounted and fixed on the upper dental arch by means of the open fixing frame with good fixing effectiveness, which eliminates the need to produce additional fixing members, and thus resolves the problem of additional fixing members hindering occlusal action.
3. The palate pad of the present invention can be custom-made, whereby the patient do swallow and uses their tongue to press up on the outer surface of the palatal augmentation palate pad, and to take mucosa side shape according to the palatal mucosa form in oral cavity. Hence, the palate pad can be adapted to match the needs of different patients, enabling the training auxiliary device of the present invention to be firmly positioned and stable within the oral cavity, thus providing better comfort when in use.
4. The present invention differs from oral appliance as swallow auxiliary devices of the prior art made from hard resin, in that the open fixing frame of the present invention is made from elastic material, such as thin thermoplastic resin plate, and the palate pad is made from soft material, such as denture soft liner, thus, the entire structure of the device is relatively soft and elastic, which provides better comfort and enables easier placement within the oral cavity when in use.
5. The present invention is aimed at different patients, and uses a dental stone cast of the upper jaw from dental impression, or 3D digital data obtained from scanning the upper dental arch and gum to first produce the open fixing frame that matches the external form of the teeth of upper dental arch, which facilitates firmly binding the open fixing frame onto the upper dental arch and reduces the sensation of having a foreign body inside the oral cavity when in use.
6. Production steps of the training auxiliary device of the present invention are relatively simple, we can make it bedside for patient, so we can quickly and appropriately meet the needs for dysphagia patient, and the importance of early intervention and training for dysphagia patient had been proved already, such as for stroke patient.

To enable a further understanding of said objectives and the technological methods of the invention herein, a brief description of the drawings is provided below followed by a detailed description of the preferred embodiments.

### BRIEF DESCRIPTION OF THE DRAWINGS

FIG. 1 is a three-dimensional external view of a training auxiliary device of the present invention not yet being placed inside the oral cavity for shaping, wherein a baseplate for a palate pad template is not yet coated with a colloid layer.

FIG. 2 is a three-dimensional external view of a training auxiliary device of the present invention ready to be placed inside the oral cavity for shaping, wherein the baseplate of the palate pad template is already coated with a colloid layer.

FIG. 3 is an external view showing a patient's upper dental arch fitted with the training auxiliary device of the present invention.

FIG. 4 is a bottom view of FIG. 3.

FIG. 5 is a schematic view depicting fitting the present invention, whereby a patient does swallow and uses tongue to press up on the palate pad template, causing the palate pad template to be molded and shaped according to the contour of the tongue elevation during swallowing and palate in the oral cavity.

FIG. 6 is another schematic view depicting fitting the present invention, whereby a patient do swallow and uses their tongue to press up on the palate pad template, causing the palate pad template to be molded and shaped according to the contour of the tongue elevation during swallowing and palate in the oral cavity.

FIG. 7 is an external view of the training auxiliary device of the present invention fitted on the patient's upper dental arch and palate with the shaped palate pad.

FIG. 8 is a schematic view of an auxiliary binding structure formed on an inner edge of an open fixing frame of the training auxiliary device of the present invention that firmly connects and fixes the palate pad.

FIG. 9 is a schematic view of another form of the training auxiliary device of the present invention, showing the open fixing frame only covering the teeth of upper dental arch and not covering the gum.

FIG. 10 is a schematic view of one of the forms of a oral appliance as auxiliary device of the prior art,

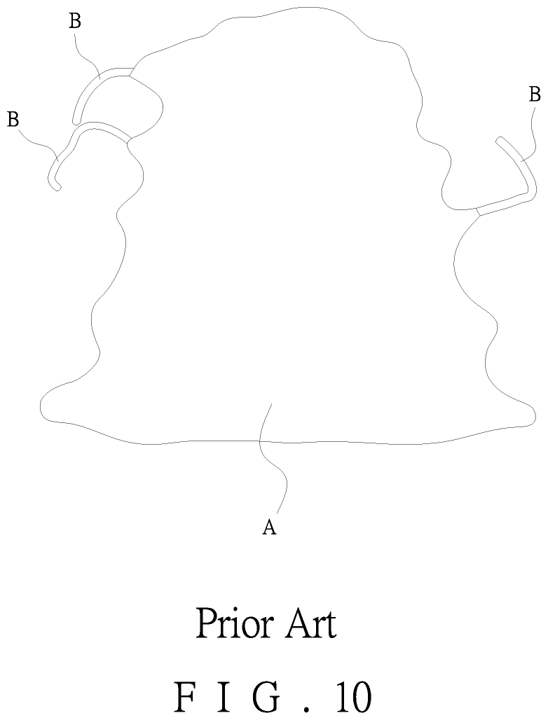

FIG. 11 is a rear side view of FIG. 10, showing a palate pad with a palatal augmentation thickness of H.

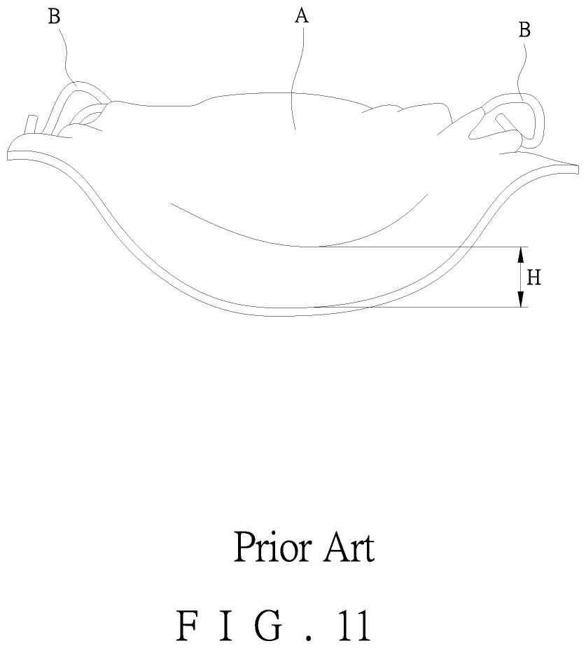

FIG. 12 is a schematic view of another form of a oral appliance as auxiliary device of the prior art.

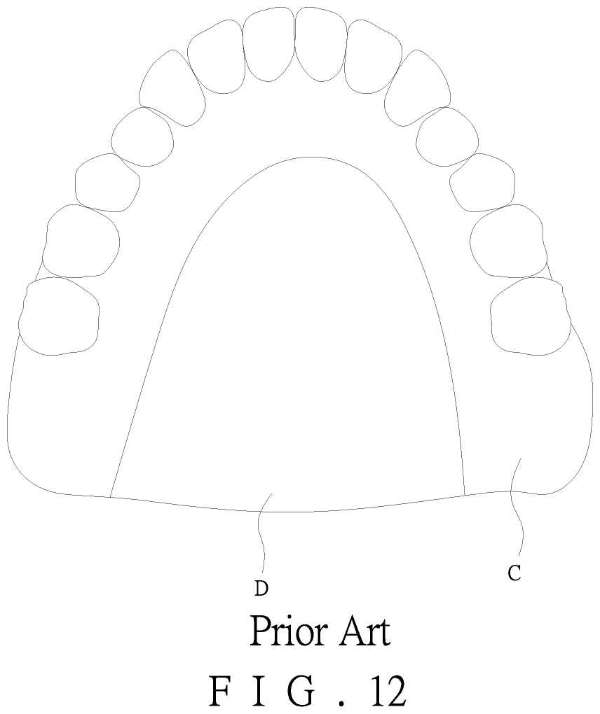

FIG. 13 is a rear side view of FIG. 12, showing a palate pad with a thickness of H′ for palatal augmentation form.

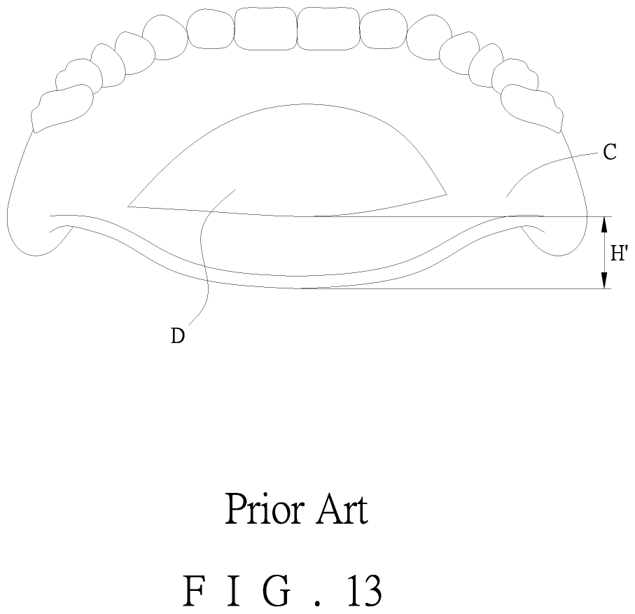

### DETAILED DESCRIPTION OF THE PREFERRED EMBODIMENTS

Based on the above-described technological characteristics of the present invention, the main effects of an oral appliance as swallow training auxiliary device with open occlusal surface and production method thereof are clearly presented in the following embodiments.

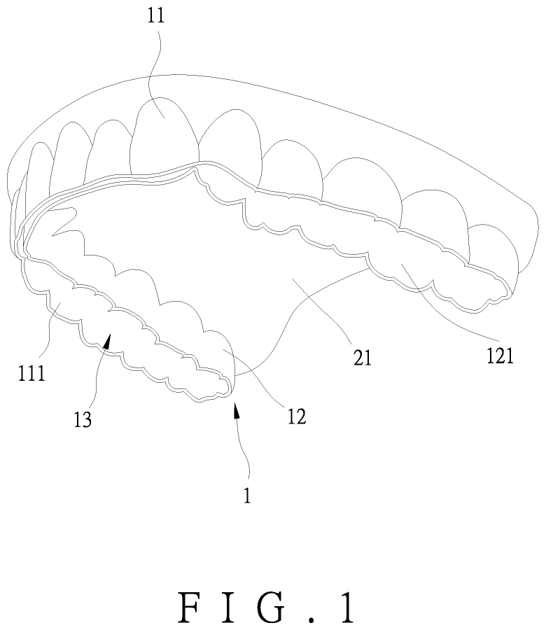

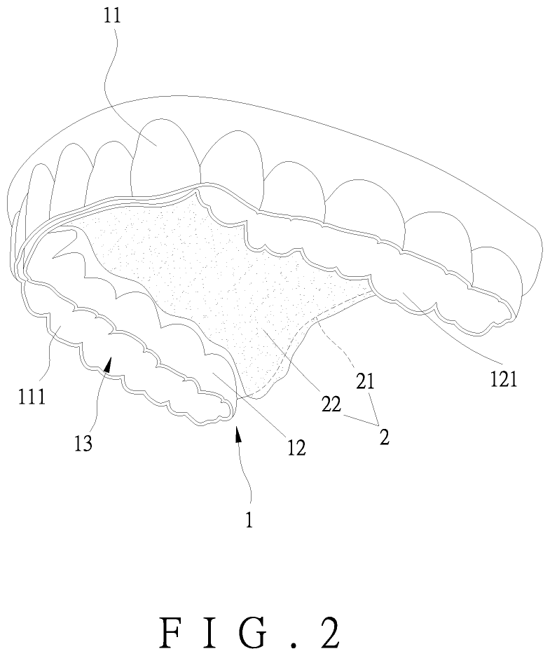

Referring to FIGS. 1, 2, and 3, which show an embodiment of a training auxiliary device of the present invention, wherein an open fixing frame (1) is produced according to the arranged configuration of an upper dental arch (10) and the shape of a gum (20) around the upper dental arch (10) in a patient's oral cavity. Accordingly, the open fixing frame (1) assumes an teeth arch form brace, which includes a mutually connected outer edge (11) and an inner edge (12), and an open space (13) that is formed between the outer edge (11) and the inner edge (12). Elastic material, such as thin thermoplastic resin plate, is used to shape and produce the open fixing frame (1) on the upper dental arch (10) and the gum (20), according to a dental stone mold, or 3D digital data of the dental arch (10) and the gum (20) is first obtained and then a 3D shape of the open fixing frame (1) is depicted to produce the open fixing frame (1), using thin thermoplastic resin plate by thermal formed or by the 3D printing technology. Accordingly, an outer edge binding surface (111) of the outer edge (11) corresponds to the arranged configuration of the external surface of the teeth of upper dental arch (10) and the shape of the external surface of the gum (20), and an inner edge binding surface (121) of the inner edge (12) corresponds to the arranged configuration of the inner surface of the teeth of upper dental arch (10) and the shape of the inner surface of the gum (20).

Then, an unsetting soft material, such as denture soft liner, is used to make an palatal augmentation palate pad, which is connected to the inner edge (12), wherein the palate pad template (2) is produced by first forming a baseplate (21) on the inner edge (12) of the open fixing frame (1). The material used for both the baseplate (21) and the open fixing frame (1) is elastic material, such as thin thermoplastic resin plate, and the two opposite surfaces of the baseplate (21) are respectively coated with a colloid layer (22), which is an unsetting soft material, such as denture soft liner.

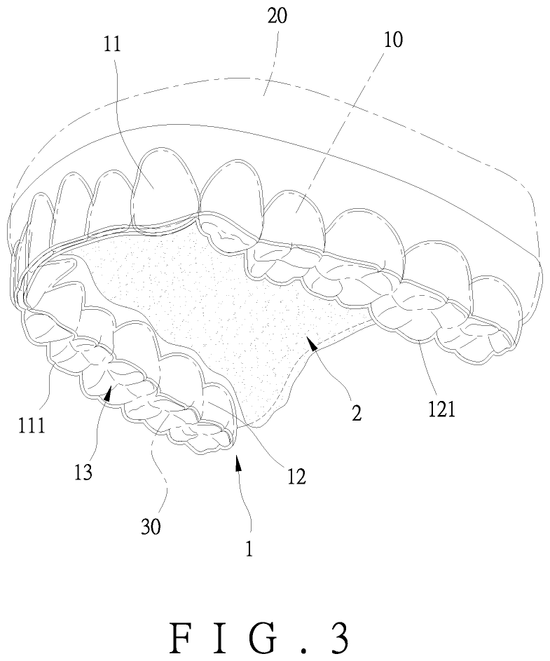

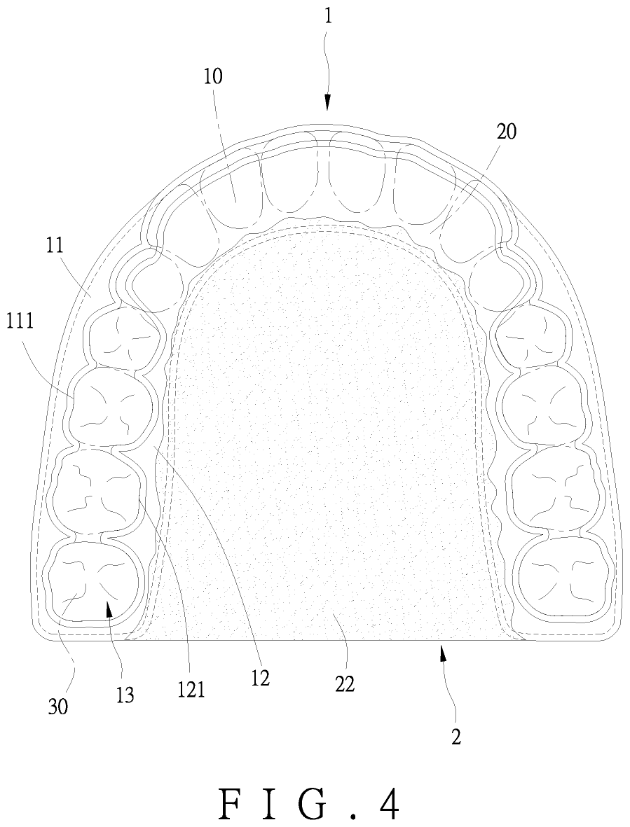

Referring to FIG. 3 and FIG. 4, when in use, the open fixing frame (1) is worn on the upper dental arch (10) in the patient's oral cavity, wherein the outer edge binding surface (111) of the outer edge (11) is tightly embrace to the external surfaces of the upper dental arch (10) and the gum (20), and the inner edge binding surface (121) of the inner edge (12) is tightly embrace to the inner lingual surfaces of teeth (10) and the gum (20). Accordingly, the open fixing frame (1) is firmly mounted onto the upper dental arch (10), which minimizes to and fro movement of the training auxiliary device when the patient do swallow training. Moreover, the open space (13) enables exposing an occlusal surface (30) of the to teeth of upper dental arch (10). Furthermore, the open fixing frame (1) is made from elastic material, such as thin thermoplastic resin plate, and the colloid layers (22) for the palate pad template (2) are made by soft material, such as denture soft liner, thus, the entire device is relatively soft and elastic, providing better comfort when in use and can realize is appropriate deformation to well match the insertion and set stable in the patient's oral cavity, even in the case patient without properly straight path of insertion for the prior art device.

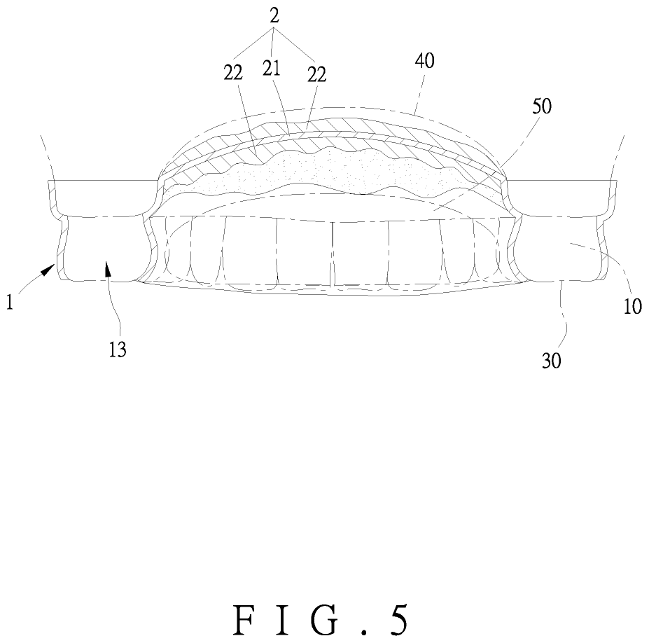

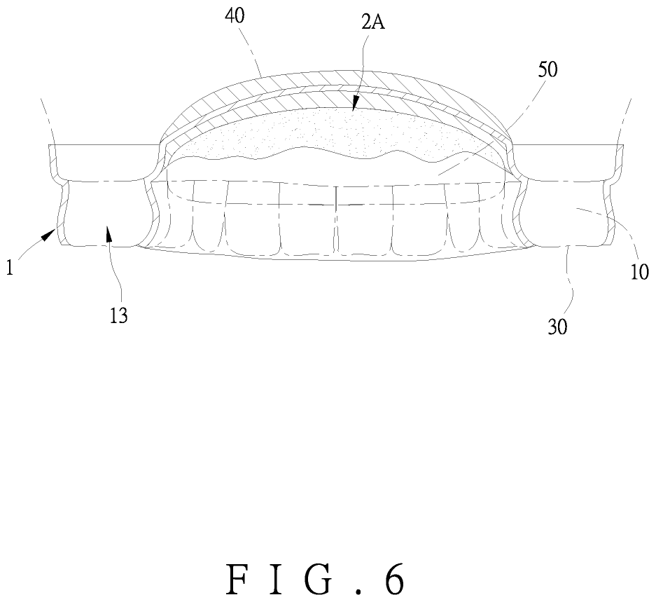

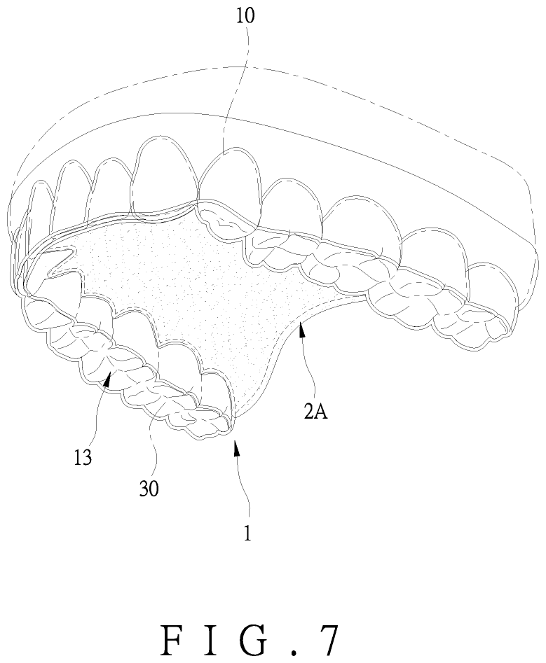

Referring to FIGS. 5, 6, and 7, the patient do swallow and uses their tongue (50) to press up on and cause the palate pad template (2) to yield to the pressing up pressure of the tongue elevation during swallowing (50) to shape a palate pad (2A), after which the patient waits for the soft material on palate pad (2A) to shape and set. Here, by way of example, the palate side of the oral cavity is an upper palate (40), and specifies the hard palate portion of the upper palate, the customized palate pad (2A) can be adapted to match the palate side shape inside the oral cavity and tongue elevation position during swallowing of different patients, thus enabling the training auxiliary device of the present embodiment to be firmly positioned within the patient's oral cavity and providing better comfort when in use.

By shortening the distance between the tongue (50) and the upper palate (40) of the patient, when the patient prepares to swallow and presses their tongue up on the palate pad (2A), it can be determined that the maximum tongue pressure of the patient has been restored to approximately 20 kPa to 30 kPa using a tongue pressure measure device, is thus enabling the patient to do swallow training if the safety to swallowing is ensured. Moreover, when the open fixing frame (1) is mounted on the upper dental arch (10), the occlusal surface (30) of the teeth of upper dental arch (10) is exposed through the open space (13), hence, the patient can still directly use the occlusal surface (30) of the teeth of upper dental arch (10) to produce occlusal action whether speaking or chewing food that is equivalent to the occlusal habit of the average person, enhance the coordination of muscle action during chewing and swallowing, increases the patient's willingness to use the training auxiliary device of the present embodiment and improves the degree of comfort when in use. Furthermore, the open fixing frame (1) embraces and is thereby fixed on the upper dental arch(10) with good fixing effectiveness, thus eliminating the need to produce additional fixing members, as well as resolving the problem of additional fixing members hindering occlusal action.

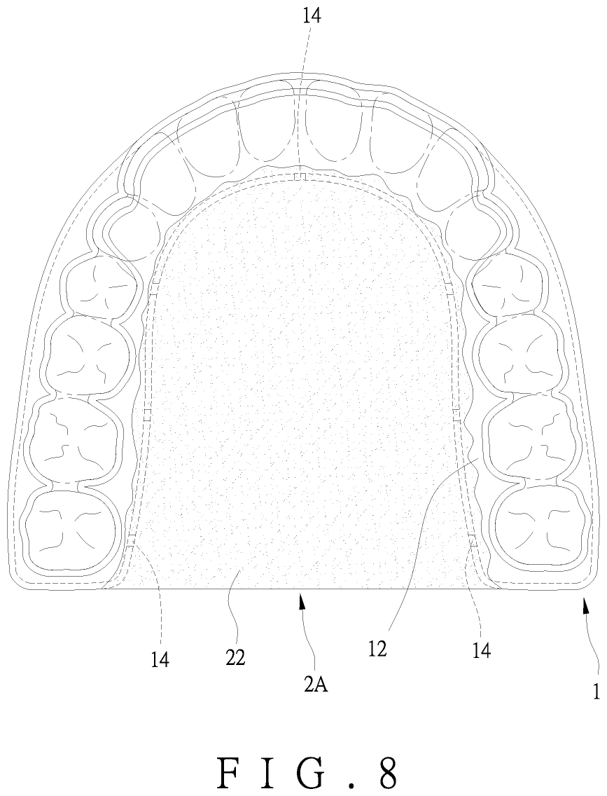

Referring to FIG. 8, in order to enable the colloid layers (22) to firmly bind to the inner edge (12) of the open fixing frame (1) without easily coming loose, an auxiliary binding structure is disposed on the inner edge (12), wherein the auxiliary binding structure of the present embodiment is a spaced arrangement of a plurality of through holes (14) is in the inner edge (12). The colloid layers (22) are embedded in the above-described through holes (14) during the process of forming thereof, which firmly bonds the colloid layers (22) to the inner edge (12) after forming the palate pad (2A), and enables extending the serviceable life of the palate pad (2A).

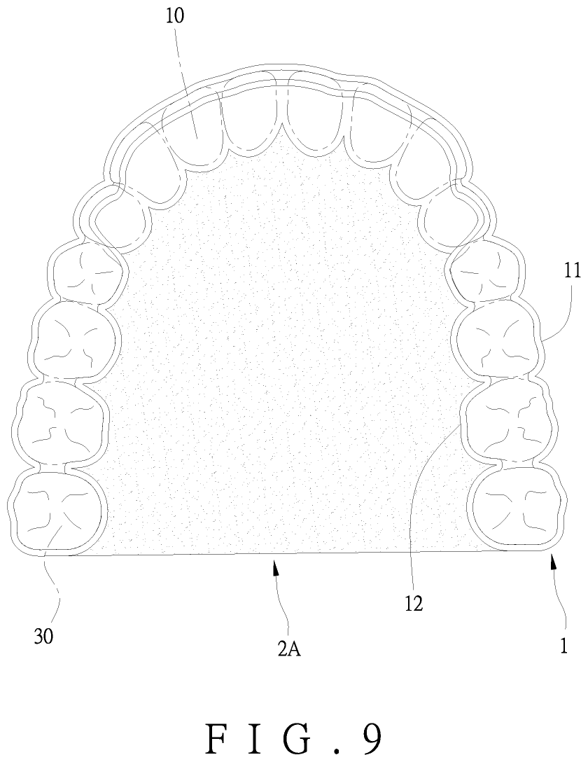

Referring to FIG. 3, the above-described open fixing frame (1) includes portions corresponding to the upper dental arch (10) and the gum (20). Thereby, if the teeth of upper dental arch are inadequately healthy and not firm enough, the open fixing frame (1) can embrace and thereby fixed on the gum(20) for more retention force, as well as the teeth of upper dental arch(10). However, referring to FIG. 9, if the teeth of upper dental arch(10) of the patient are adequately healthy and firm enough, the open fixing frame (1) can also be worn to just embrace the teeth of upper dental arch(10), whereby the outer edge (11) and the inner edge (12) of the open fixing frame (1) only correspond to the to arranged configuration of the teeth of upper dental arch(10) and is only mounted on the teeth of upper dental arch (10), exposing the occlusal surface (30) thereof. Accordingly, the sensation of having a foreign body on the gum (20), as shown in FIG. 3, is reduced and degree of comfort using the open fixing frame (1) is improved.

In summary, the above description of the embodiments provides a clear understanding of the operational procedure and the effectiveness achieved by the present invention. However, it is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. An oral appliance as swallow training auxiliary device with open occlusal surface, comprising:
an open fixing frame, an arch form teeth brace, the brace having an outer edge and an inner edge mutually connected, wherein an open space is defined between the outer edge and the inner edge enables exposing occlusal surface of upper dental arch; and a palate pad, corresponding in shape to a palate side and tongue elevation during swallowing in an oral cavity, the palate pad being fixed to the inner edge.

2. The device according to claim 1, wherein the outer edge includes an outer edge binding surface corresponding to an arranged configuration of an external surface of the teeth of upper dental arch, and the inner edge includes an inner edge binding surface corresponding to an arranged configuration of an inner surface of the teeth of upper dental arch.

3. The device according to claim 1, wherein the open fixing frame is made from elastic material, such as thin thermoplastic resin plate.

4. The device according to claim 1, wherein the palate pad is made from soft material, such as denture soft liner.

5. The device according to claim 1, wherein an auxiliary binding structure is disposed on the inner edge, and the palate pad is fixed to the inner edge by means of the auxiliary binding structure.

6. The device according to claim 5, wherein the auxiliary binding structure is provided with several through holes.

7. The device according to claim 1, wherein the palate pad includes a baseplate and colloid layers respectively coated on two opposite surfaces of the baseplate.

8. The device according to claim 7, wherein the open fixing frame and the baseplate are made from elastic material, such as thin thermoplastic resin plate, and the colloid layers are made from soft material, such as denture soft liner.

9. The device according to claim 1, wherein the open fixing frame is configured to be only mounted on the teeth of upper dental arch.

10. The device according to claim 9, wherein the open fixing frame is configured to be mounted on gum nearby the upper dental arch as well as the teeth of upper dental arch.

11. A production method for an oral appliance as swallow training auxiliary device with open occlusal surface, comprising the following steps: Producing an open fixing frame according to an arranged configuration of an upper dental arch, wherein open fixing frame includes an outer edge and an inner edge mutually connected, with an open space being formed therebetween that enables exposing an occlusal surface of the upper dental arch, and producing a palate pad to match on the inner edge palate side and tongue elevation position during swallowing in an oral cavity.

12. The production method according to claim 11, wherein the open fixing frame is produced according to the arranged configuration of the upper dental arch and a shape of gum around upper dental arch.

13. The production method according to claim 11, wherein the open fixing frame is produced by applying elastic material, such as thin thermoplastic resin plate on a dental mold with upper dental arch so as to be shaped according thereto.

14. The production method according to claim 11, wherein the open fixing frame is produced by firstly obtaining 3D digital data of the upper dental arch and gum, then depicting and printing out the open fixing frame with a 3D printing technology.

15. The production method according to claim 11, wherein an unsetting soft material, such as denture soft liner is used to produce palate pad that is connected to the inner edge; after wearing the open fixing frame with the palate pad template, a patient do swallow and uses his tongue to push up on and cause the palate pad template to be shaped and then form the palate pad according to the palate side and tongue elevation during swallowing in the oral cavity, after which the palate pad is setting.

16. The production method according to claim 15, wherein the palate pad template is produced by first forming a baseplate on the inner edge of the open fixing frame, and then the two opposite surfaces of the baseplate are coated with the unsetting soft material, such as denture soft liner.
